(19) United States
(12) Patent Application Publication
(10) Pub. No.: US 2011/0047653 A1
(43) Pub. Date: Feb. 24, 2011

(54) CULTIVARS OF THE POPPY PAPAVER BRACTEATUM THAT PRODUCE SEED CAPSULES AND THEBAINE IN THE FIRST GROWING SEASON

(75) Inventor: Jarrod David Ritchie, Launceston (AU)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: TPI Enterprises Ltd., Cressy (AU)

(21) Appl. No.: 12/735,548

(22) PCT Filed: Jan. 22, 2009
(86) PCT No.: PCT/AU2009/000065

§ 371 Date: Nov. 1, 2010

(30) Foreign Application Priority Data
Jan. 24, 2008 (AU) 2008900325

(51) Int. Cl. A01H 5/00; A01H 5/10; C12P 17/18; C07D 489/02; C12N 5/04; C08L 9/04

(52) U.S. Cl. 800/298; 435/119; 546/44; 435/410; 524/501

(57) ABSTRACT

The present invention relates to poppy cultivars and their use to produce thebaine. In specific forms, the present invention relates to cultivars of Papaver bracteatum that produce thebaine in the first growing season after germination.

### PRIORITY CLAIM

The present application claims priority to Australian provisional patent application 2008900325, the contents of which are hereby incorporated by reference.

### FIELD OF THE INVENTION

The present invention relates to poppy cultivars and their use to produce thebaine. In specific forms, the present invention relates to cultivars of Papaver bracteatum that produce thebaine in the first growing season after germination.

### BACKGROUND OF THE INVENTION

Opium poppy is commercially cultivated in a number of countries under regulatory control. The latex obtained by the incision of unripe capsules is known as opium and is the source of several pharmacologically important alkaloids. Morphine, codeine, thebaine, narcotine and papaverine are the most important alkaloids produced by the plant, and are exploited by the pharmaceutical industry as analgesics, antitussives and antispasmodics.

On a commercial scale, however, poppy straw and straw concentrate (rather than latex) are the most commonly used raw materials for the production of morphine and other poppy-derived alkaloids. For example, more than 50% of the world's morphine is now manufactured from poppy straw or poppy straw concentrate.

Thebaine is one of the phenanthrene alkaloids contained in opium poppies. Thebaine is dehydromorphine 3,6-dimethyl ether, which has the structure set out below:

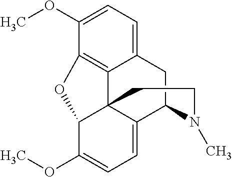

The chemical structure of thebaine is superficially similar to both morphine and codeine, but produces stimulatory effects rather than depressant effects. Due to the pharmacological properties of thebaine, it is not generally used therapeutically. However, thebaine may be converted into a variety of pharmaceutical agents including oxycodone, oxymorphone, nalbuphine, naloxone, naltrexone, buprenorphine and etorphine.

Commercially viable cultivars of naturally occurring opium poppy that produce significant quantities of thebaine were unavailable until the development of the ‘Norman poppy’, a mutant of Papaver somniferum (U.S. Pat. No. 6,723,894). This mutant poppy is also an annual, as are other cultivars of Papaver somniferum.

Prior to the development of this variety, crops of Papaver somniferum were sprayed with growth regulation chemicals to interrupt alkaloid biosynthetic pathways at an earlier stage, and thus enhance the quantity of thebaine.

Given an increasing world demand for thebaine derived pharmaceuticals, a significantly higher yielding thebaine producing poppy would represent a distinct advantage, particularly if the poppy were also of the perennial type, avoiding the necessity to replant every year, therefore saving farmers the necessity of annual replanting.

Australia produces a significant portion (approximately 30% in 2006) of the world's poppy straw concentrates for morphine production. Furthermore, the Australian State of Tasmania is the world's largest producer of opium alkaloids for the pharmaceutical market. The area sown to poppies in Tasmania was close to 8,000 ha in 2006.

However, a significant problem affecting the cultivation of poppies for thebaine production in at least some poppy growing regions (including Tasmania, Australia) is that high thebaine-yielding varieties, particularly of the species Papaver bracteatum, do not produce seed capsules and thebaine in the first growing season after seed germination. Thus, in these regions, commercially viable thebaine poppies cannot be harvested until at least the second growing season after sowing a poppy crop.

Accordingly, there is a need for poppy cultivars that produce extractable quantities of thebaine in the first growing season after germination, particularly cultivars of Papaver bracteatum that produce extractable quantities of thebaine in the first growing season after germination in at least temperate poppy-growing regions, such as Tasmania, Australia.

Reference to any prior art in this specification is not, and should not be taken as, an acknowledgment or any form of suggestion that this prior art forms part of the common general knowledge in any country.

### SUMMARY OF THE INVENTION

The present invention is predicated, in part, on the breeding of poppy cultivars that produce seed capsules and an extractable quantity of thebaine in the first growing season after germination.

In some embodiments, the poppy plants of the present invention which produce seed capsules and an extractable quantity of thebaine in the first growing season after germination are of the species Papaver bracteatum.

In some embodiments, the plants of the present invention produce seed capsules and an extractable quantity of thebaine in the first growing season after germination in at least a temperate poppy growing region. In yet another embodiment, the plants of the present invention produce seed capsules and an extractable quantity of thebaine in the first growing season after germination in at least a temperate poppy growing region in the Southern hemisphere.

In some embodiments, the plants of the present invention produce seed capsules and an extractable quantity of thebaine in the first growing season after germination in Tasmania, Australia.

The present invention also provides reproductive material derived from the plant of the first aspect of the invention, or a progeny, mutant or derivative thereof.

In another aspect, the present invention also provides straw produced from a plant of the first aspect of the invention, or a progeny, mutant or derivative thereof.

In another aspect, the present invention also provides a straw concentrate produced from a plant of the first aspect of the invention or a progeny, mutant or derivative thereof.

In yet another aspect, the present invention also provides latex derived from the plant of the first aspect of the invention or a progeny, mutant or derivative thereof.

The present invention also provides a stand of stably reproducing poppy plants from which an extractable quantity of thebaine may be produced in the first growing season after germination of the poppy plants, the stand comprising one or more plants according to the first aspect of the invention, or a progeny, mutant or derivative thereof.

The present invention also extends to a method of producing thebaine, the method comprising growing a plant of the first aspect of the invention, or a progeny, mutant or derivative thereof, such that the plant produces seed capsules and thebaine in the first growing season after germination; and harvesting thebaine from the poppy plant or a part thereof.

In a yet further aspect, the present invention provides an isolated cell derived from the plant of the first aspect of the invention or a progeny, mutant or derivative thereof.

The present invention also extends to an in-vitro culture including one or more of the cells described above. Exemplary “in-vitro cultures” contemplated herein include, for example, callus cultures and suspension cultures which include one or more of the cells of the invention. Generally, the in-vitro cultures of the present invention may be used to produce thebaine.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### DESCRIPTION OF EXEMPLARY EMBODIMENTS

It is to be understood that the following description is for the purpose of describing particular embodiments only, and is not intended to be limiting with respect to the above description.

In a first aspect, the present invention provides a poppy plant which produces seed capsules and an extractable quantity of thebaine in the first growing season after germination.

As referred to herein, a “poppy plant” should be understood to refer to any thebaine-producing member of the genus Papaver. Exemplary species within the genus Papaver include the Papaver species described in Table 1.

TABLE 1
Exemplary poppy plants within the genus Papaver

| Species | Common Name | Typical Description |
|---|---|---|
| Papaver somniferum | Oil Poppy | Papaver somniferum or Oil Poppy grows to a |
| | | height of up to 1.5 meters. It has 4 petals which |
| | | are pale pink with a dark basal blotch. The capsules |
| | | are spherical with a flat cap and are 20-40 mm in |
| | | diameter. |
| Papaver bracteatum | Persian Poppy | Papaver bracteatum or Persian Poppy grows to a |
| | | height of up to 1.5 meters. It has 6 petals which |
| | | are blood red in colour with a dark basal blotch. |
| | | The capsules are 40 mm diameter with a concave |
| | | cap and persistent bracts. |
| Papaver rhoeas | Field Poppy | Papaver rhoeas or Field Poppy grows to a height |
| | | of up to 600 mm. It has 4 petals which are red and |
| | | may have a dark basal blotch. The capsules are |
| | | elongated and are 5-10 mm in diameter and |
| | | 15-20 mm in length. |
| Papaver pseudo- | Pseudo-oriental | Papaver pseudo-orientale or Pseudo-oriental Poppy |
| orientale | Poppy | grows to a height of 400-600 mm. It has 4-6 petals |
| | | which are deep orange “scarlet” with a part blotch. |
| | | The capsules can be up to 25 mm long with a flat |
| | | disc. |
| Papaver orientale | Oriental Poppy | Papaver orientale or Oriental Poppy grows to a |
| | | height of 300-700 mm. It has 4 petals which are |
| | | pale orange in colour with no blotch. The capsules |
| | | are 20 mm long with no bracts. |
| Papaver hybridum | Rough Poppy | Papaver hybridum or Rough Poppy grows to a |
| | | height of up to 500 mm. It has 4 petals which are |
| | | crimson in colour with a dark basal blotch. The |
| | | capsules are 15 mm long, oval in shape and |
| | | bristly. |
| Papaver dubium | Long-head | Papaver dubium or Long-head Poppy grows to a |
| | Poppy | height of up to 600 mm. It has 4 petals which are |
| | | red in colour and overlapping with no blotch. The |
| | | capsules are elongated, 15-20 mm long, and |
| | | 5-10 mm in diameter. |
| Papaver argemone | Pale Poppy | Papaver argemone or Pale Poppy grows to a height |
| | | of up to 500 mm. It has 4 petals which are dark |
| | | crimson in colour with a dark basal blotch. The |
| | | capsules are 10-15 mm long, elongated and bristly. |

Also, in some embodiments (as described later) the term “poppy” should also be understood to refer to thebaine-producing hybrid plants wherein at least one of the parents is a plant of the genus Papaver.

In some embodiments, the term “poppy plant” refers to a perennial poppy plant. As referred to herein, a “perennial” poppy plant encompasses poppy plants that produce seed capsules and an extractable quantity of an alkaloid such as thebaine for more than one growing season without the need to replant.

In some embodiments of the invention, the term “poppy plant” refers to a poppy plant of the species Papaver bracteatum or a hybrid poppy plant wherein at least one of the parents of the hybrid plant is a poppy plant of the species Papaver bracteatum.

As described below, the term “poppy plant” may also include derivatives and mutants of poppy plants.

In addition, the term poppy plant may refer to a whole poppy plant, but may also refer to a part of a poppy plant, including, for example, reproductive material (such as seeds) derived from a poppy plant; a cell, tissue or organ derived from a poppy plant; and the like.

As set out above, the poppy plants of the present invention produce seed capsules and an extractable quantity of thebaine in the first growing season after germination.

As referred to herein, the term “thebaine” should be understood as dehydromorphine 3,6-dimethyl ether, which has the structure set out below:

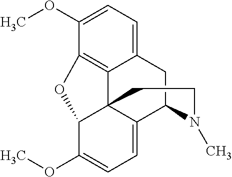

“An extractable quantity of thebaine” should be understood to be any amount of thebaine that may be extracted from the plant, or any part thereof (such as straw or latex).

Methods for the extraction and detection of thebaine are known in the art. For example, thebaine and related alkaloids may be extracted from dried poppy heads and poppy straw using the methods described in French patent 748308, and various modifications thereof. In general, thebaine is extracted from poppy seed capsules using either aqueous acidic extraction conditions or aqueous alkaline extraction conditions to which various water miscible organic solvents have been added.

In some embodiments of the invention “the extractable quantity of thebaine” is a relatively high concentration of thebaine within the plant or a part thereof. In these embodiments, the extractable quantity of thebaine may comprise, for example, anhydrous thebaine comprising at least 1.0% by weight of the seed capsules by weight of a seed capsule of the plant when dried to a moisture level of 9-16% as determined by a Loss On Drying (LOD) analysis.

In further embodiments, the extractable quantity of anhydrous thebaine may comprise, for example, at least 1.5%, at least 2.0%, at least 2.5%, at least 3.0%, at least 3.5%, at least 3.6%, at least 3.7% or at least 3.8% by weight of the seed capsules by weight of a seed capsule of the plant when dried to a moisture level of 9-16% as determined by a Loss On Drying (LOD) analysis.

The concentration of thebaine in P. bracteatum seed capsules may by assessed using any suitable method. For example, reference is made to the methods disclosed by Wu and Dobberstein (Journal of Chromatography A 140(1): 65-70, 1977) and Shou et al. (Rapid Communications in Mass Spectrometry 16: 1613-1621, 2002).

Production of seed capsules and an extractable quantity of thebaine “in the first growing season after germination” should be understood as the plants producing seed capsules and an extractable quantity of thebaine within one year of seed germination.

In some embodiments, the plants produce flowers within about 160 days after sowing, and/or are suitable for harvesting and thebaine extraction at about 330 days after sowing.

In some embodiments, the plants of the present invention which produce seed capsules and an extractable quantity of thebaine in the first growing season after germination in at a least temperate poppy growing region.

As referred to herein, “a temperate poppy growing region” should be understood as a region in which poppies may be cultivated which is located at a latitude that lies between the tropics and the polar circles. Specifically, the north temperate zone extends from the Tropic of Cancer at about 23.5 degrees north latitude to the Arctic Circle at about 66.5 degrees north latitude. The south temperate zone extends from the Tropic of Capricorn at about 23.5 degrees south latitude to the Antarctic Circle at about 66.5 degrees south Latitude.

In some embodiments, the temperate poppy growing region may include a region where the poppies of the present invention will grow which is at one or more latitudes of about 34°, about 35°, about 36°, about 37°, about 38°, about 39°, about 40°, about 41°, about 42°, about 43°, about 44°, about 45°, about 46°, about 47°, about 48°, about 49°, and/or about 50°.

In some embodiments, the plants of the present invention produce seed capsules and an extractable quantity of thebaine in the first growing season after germination in at least a temperate poppy growing region in the Southern hemisphere. Thus, in some embodiments, the plants of the present invention may produce seed capsules and an extractable quantity of thebaine in the first growing season after germination at one or more latitudes of about 34° S, about 35° S, about 36° S, about 37° S, about 38° S, about 39° S, about 40° S, about 41° S, about 42° S, about 43° S, about 44° S, about 45° S, about 46° S, about 47° S, about 48° S, about 49° S, and/or about 50° S.

Reference herein to a latitude “about” a particular latitude should be understood to encompass latitudes ±0.5° of the defined latitude. In addition, reference to “one or more latitudes” should be understood to encompass discreet ranges of latitudes bounded by any pairs of the specific latitudes mentioned above.

In some embodiments, the plants of the present invention produce seed capsules and an extractable quantity of thebaine in the first growing season after germination at a latitude of between about 40° S and about 44° S.

In some embodiments, the plants of the present invention produce seed capsules and an extractable quantity of thebaine in the first growing season after germination in Tasmania, Australia or a region having substantially equivalent climate.

The average rainfall and temperatures in Tasmania vary considerably across the state. However, in the main poppy growing regions in the north-west and midlands of the state, the typical rainfall is in the range of 600 to 800 mm, and the minimum/maximum temperatures are 2° C. and 12° C. respectively for July and 12° C. and 24° C. for January and February. Typically, in Tasmanian poppy growing regions, poppies are planted in March and the dried poppy heads are harvested in the following February.

Reference herein to a “region having substantially equivalent climate” refers to a region having equivalent day length, rainfall and/or seasonal temperature minima and maxima such that conditions under which poppies may be grown are equivalent to those in Tasmania, Australia.

The poppy plants of the present invention may have either a dehiscent (open) seed capsule or an indehiscent (closed) seed capsule at seed maturity.

Wild type P. bracteatum, and most other Papaver species, form pores of dehiscence in the seed capsule at seed maturity, which allows the mature seed in the seed capsule to be released from the seed capsule. However, in some embodiments of the present invention, the poppy cultivar may have seed capsules that have reduced or no pores of dehiscence at the time the seed in the seed capsule has matured. Thus, the trait of an indehiscent or closed seed capsule at maturity leads to the retention of mature seed in the seed capsule in cultivars having the trait.

One significant advantage of the indehiscent or closed seed capsule at maturity trait is that seed is retained on harvesting, and the dispersal of seed is substantially more controlled, minimising excessive plant density with perennial species. Additionally, the seed from cultivars having an indehiscent seed capsule can be readily collected from the seed capsule and further utilised.

Two exemplary poppy cultivars of the present invention have been deposited in accordance with the provisions of the Budapest Treaty on the International Recognition of the Deposit of Microorganisms for the Purposes of Patent Procedure.

Seed from the first exemplary cultivar, referred to herein as PB-1, having an indehiscent or closed seed capsule at maturity, has been deposited at the National Collections of Industrial and Marine Bacteria Limited (NCIMB) on 3 Jan. 2008 under accession number NCIMB 41534.

Accordingly, in one specific embodiment, the plant of the first aspect of the invention is produced from seed deposited with the National Collections of Industrial and Marine Bacteria Limited under accession number NCIMB 41534; or a progeny, mutant or derivative of said plant that retains the ability to produces seed capsules and an extractable quantity of thebaine in the first growing season after germination.

Seed from the second exemplary cultivar, referred to herein as PB-2, having a dehiscent or open seed capsule at maturity, has been deposited at the National Collections of Industrial and Marine Bacteria Limited (NCIMB) on 3 Jan. 2008 under accession number NCIMB 41535.

Accordingly, in another specific embodiment, the plant of the first aspect of the invention is produced from seed deposited with the National Collections of Industrial and Marine Bacteria Limited under accession number NCIMB; or a progeny, mutant or derivative of said plant that retains the ability to produces seed capsules and an extractable quantity of thebaine in the first growing season after germination.

The present invention also provides a progeny plant, wherein the progeny plant has a plant of the first aspect of the invention as a parent, and wherein said progeny plant produces an extractable quantity of thebaine in the first growing season after germination.

As referred to herein, a “progeny” plant may be any plant for which a plant of the first aspect of the invention is at least one of the parents. Thus, the progeny plant may be a plant resulting from self-fertilisation of a plant of the first aspect of the invention; a plant resulting from a non-self fertilisation wherein a plant of the first aspect of the invention is the male or female parent; an intrageneric hybrid plant wherein one of the parents is a plant of the first aspect of the invention and the other parent is a plant within the genus Papaver; an intergeneric hybrid plant wherein one of the parents is a plant of the first aspect of the invention and the other parent is a plant of a genus other than Papaver; an asexually produced progeny of a plant of the first aspect of the invention, such as vegetatively reproduced progeny or progeny produced by apomixis.

The present invention also provides a mutant or derivative of the poppy plant of the first aspect of the invention wherein the mutant or derivative produces seed capsules an extractable quantity of thebaine in the first growing season after germination.

A “mutant or derivative” of the subject poppy plants should be understood to encompass, for example, any spontaneous or induced mutant, breeding progeny or genetically modified form of the deposited strains which retains the ability to produces seed capsules and an extractable quantity of thebaine in the first growing season after germination.

Mutagenisation techniques that may be used to generate derivatives or mutants of the deposited poppy cultivars include, for example, chemical mutagenesis (eg. EMS mutagenesis), ionising radiation-induced mutagenesis (eg. X-ray mutagenesis, γ-ray mutagenesis and UV mutagenesis), genetic insertion mutagenesis methods (eg. transposon mutagenesis or T-DNA mutagenesis) and the like. Techniques for the production of mutagenized seed are well known in the art. For example, methods of seed mutagenesis as well as chemical mutagens suitable for use in these methods are described in, for example, The Manual on Mutation Breeding, 2nd ed. (I.A.E.A., Vienna, 1977) or in Plant Breeding, Principles and Prospects (Chapman and Hall, London, 1993). Example mutagens include ethyl methanesulfonate (EMS), diepoxybutane (DEB) ethyl-2-chloroethyl sulphide, 2-chloroethyl-dimethylamine, ethylene oxide, ethyleneimine, dimethyl sulphonate, diethyl sulphonate, propane sulphone, beta-propiolactone, diazomethane, N-methyl-N-nitrosourethane, acridine orange and sodium azide. An example of ionising radiation induced mutagenesis, specifically X-ray mutagenesis is described and compared to EMS mutagenesis by Filippetti et al. (Euphytica 35: 49-59, 1986).

In some embodiments, the progeny, mutant or derivative plants of the present invention produce seed capsules and an extractable quantity of thebaine in the first growing season after germination in one or more of the geographical locations hereinbefore described.

The present invention also provides reproductive material derived from the plants described herein. The term “reproductive material” should be understood as any material from which a plant may be reproduced. For example, “reproductive material” may include seeds, flowers, cuttings, ovaries, ovules, embryo sacs, egg cells, anthers, pollen, regenerable de-differentiated plant tissue such as callus, embryogenic callus or suspension culture, isolated plant embryos and the like.

In some embodiments, the reproductive material comprises a seed. As referred to herein, a plant “seed” should be understood to refer to a mature or immature plant seed. As such, the term “seed” includes, for example, immature seed carried by a maternal plant or seed released from the maternal plant. The term “seed” should also be understood to include any seed plant sporophyte between the developmental stages of fertilisation and germination. However, in some embodiments, the term seed refers to a mature plant seed.

In one specific embodiment, the seed comprises the seed deposited with the National Collections of Industrial and Marine Bacteria Limited under accession number NCIMB 41534, or a mutant or derivative thereof. In another specific embodiment, the seed comprises the seed deposited with the National Collections of Industrial and Marine Bacteria Limited under accession number NCIMB 41535, or a mutant or derivative thereof.

The above embodiments of the invention refer to “mutants” and “derivatives” of the deposited seed. A mutant or derivative of the deposited poppy seed should be understood to encompass, for example, any spontaneous or induced mutant seed, seed from breeding progeny or genetically modified seed, which can produce a poppy plant that retains the ability to produce seed capsules and an extractable quantity of thebaine in the first growing season after germination.

In another aspect, the present invention also provides straw produced from a plant of the first aspect of the invention, or a progeny, mutant or derivative thereof.

As defined herein the “straw” of a poppy plant includes fresh or dried tissue of a poppy plant. This tissue may include all or part of the plant, such as root tissue, shoot tissue, floral tissue or a seed capsule. In some embodiments, poppy “straw” includes fresh or dried poppy plant tissue which includes a mature seed capsule. The contemplated seed capsule may include seeds, or may be a capsule in which the seeds have been removed.

Methods for poppy straw processing to obtain alkaloids are well known in the art. However, by way of example, reference is made to French patent 748308 which discloses one example of a method for alkaloid extraction from poppy straw.

A solvent (for example, water or a super critical fluid, such as supercritical CO₂) may also be used to extract alkaloids from straw. Extracts produced according to such methods include “straw concentrates”.

Thus, in another aspect, the present invention also provides a poppy straw concentrate produced from a plant of the first aspect of the invention or a progeny, mutant or derivative thereof.

The term “poppy straw concentrate” should be understood to include any material arising when poppy straw has entered into a process for the concentration of its alkaloids. Not inconsistent with the foregoing definition, “poppy straw concentrates” should also be understood to include any crude or purified extracts of poppy straw in either liquid, solid or powder form which contain one or more phenanthrene alkaloids of an opium poppy.

When in liquid form, poppy straw concentrates may be further concentrated from a crude extract. Such concentrates may be either liquid concentrates, wherein a portion of the solvent has been removed, or powder form concentrates which result from removing substantially all of the solvent used for extraction of the poppy straw.

A poppy straw concentrate, as defined herein, may include all of the alkaloids that may be extracted from a poppy straw, or may include a subset of the extractable alkaloids. Generally, a poppy straw concentrate of the present invention at least includes thebaine.

In another aspect, the present invention also provides latex derived from the plant of the first aspect of the invention or a progeny, mutant or derivative thereof.

Methods for obtaining latex from a poppy plant, including Papaver bracteatum, are well known in the art. Generally, latex may be obtained by incising an immature seed capsule of the plant, from which latex is exuded.

In another aspect, the present invention also provides a stand of stably reproducing poppy plants from which an extractable quantity of thebaine may be produced in the first growing season after germination of the poppy plants, the stand comprising one or more plants according to the first aspect of the invention, or a progeny, mutant or derivative thereof.

In yet another aspect, the present invention also provides a method of producing thebaine, the method comprising growing a plant of the first aspect of the invention, or a progeny, mutant or derivative thereof, such that the plant to produces seed capsules and thebaine in the first growing season after germination; and harvesting thebaine from the poppy plant or a part thereof.

In various embodiments, the method of the present invention comprises a method for producing thebaine in one or more of the geographical locations hereinbefore described.

In a yet further aspect, the present invention provides an isolated cell, tissue or organ derived from the plant of the first aspect of the invention or a progeny plant thereof.

The present invention also extends to an in-vitro culture including one or more of the cells described above. Exemplary “in-vitro cultures” contemplated herein include, for example, callus cultures, embryogenic callus cultures, embryo cultures, plantlet cultures and suspension cultures which include one or more of the cells of the invention. Techniques for the establishment and maintenance of plant cell or tissue cultures are well known in the art. In this regard, reference is made to ‘Plant Tissue Culture: An Alternative for Production of Useful Metabolites’ (FAO Agricultural Services Bulletin No. 108, Food and Agriculture Organization of the United Nations Rome, 1994).

In some embodiments, a thebaine-producing in-vitro culture of Papaver bracteatum may be established using the method described in U.S. Pat. No. 4,114,314, with the modification that cells derived from the plants of the present invention are used instead of those disclosed in U.S. Pat. No. 4,114,314.

Generally, the in-vitro cultures of the present invention may be used to produce thebaine. The production of thebaine by the culture may be de novo production, or may be via the conversion of a complex substrate, such as an intermediate in the thebaine biosynthetic pathway, for example, (S)-Reticuline.

The present invention is further described by the following non-limiting examples:

### BRIEF DESCRIPTION OF THE FIGURES

FIG. 1 is a photograph showing the seed capsules of Papaver bracteatum PB-1 and Papaver bracteatum PB-2. Papaver bracteatum PB-1, which has an indehiscent or closed seed capsule at maturity is shown in the two plants to the right of the photograph, while Papaver bracteatum PB-2, which has a dehiscent or open seed capsule at maturity, is shown in the two plants to the left of the photograph.

### EXAMPLE 1

### Morphological Description of Papaver bracteatum PB-1 and Papaver bracteatum PB-2

Presented below are selected morphological characteristics of two bred poppy cultivars, Papaver bracteatum PB-1 and Papaver bracteatum PB-2.

Each of the cultivars has substantially identical morphological characteristics other than that cultivar PB-1 comprises an indehiscent seed capsule, while cultivar PB-2 comprises a dehiscent seed capsule. A summary of the morphological characteristics of the cultivars is shown below:

| | |
|---|---|
| Plant height at full maturity: | 1.2 to 1.5 meters |
| Peduncle length: | 50-80 cm, |
| Peduncle colour | green, light brown later prior to harvest. |
| Number of capsules: | 1-17 capsules per plant. |
| Capsule dehiscence at | PB-1-indehiscent |
| maturity: | PB-2-dehiscent |
| Capsule size: | 1.4-4.7 g |
| % total weight seed: | 39-54% |
| LOD: | 9-12% |
| Anhydrous Assay: | 2-5% |
| Mass of one seed: | 0.00023 g |
| Seeds per capsule: | 9,000-12,000 |
| Days to flowering: | approx 160 days. |
| Days to harvest: | approx 330 days. |

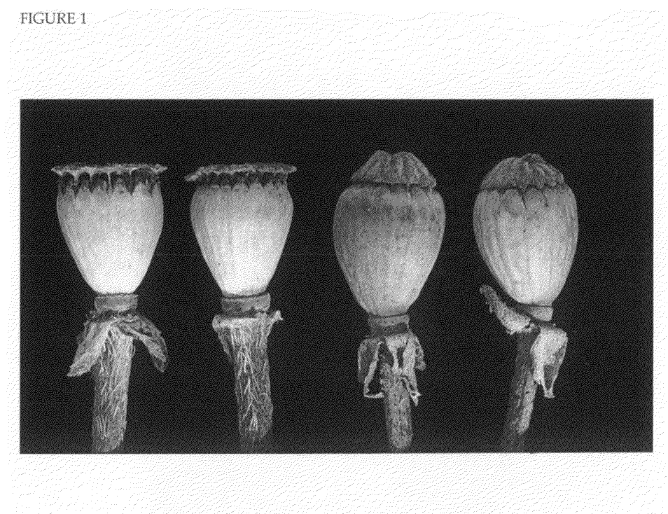

A photograph showing the differences in the seed capsule between Papaver bracteatum PB-1 and Papaver bracteatum PB-2 is shown in FIG. 1.

Seeds of Papaver bracteatum PB-1 have been deposited at the National Collections of Industrial and Marine Bacteria Limited (NCIMB) on 3 Jan. 2008 under accession number NCIMB 41534.

Seeds of Papaver bracteatum PB-2 have been deposited at the National Collections of Industrial and Marine Bacteria Limited (NCIMB) on 3 Jan. 2008 under accession number NCIMB 41535.

### EXAMPLE 2

### Thebaine Production in Papaver bracteatum PB-1 and Papaver bracteatum PB-2

Seeds of Papaver bracteatum PB-1 and Papaver bracteatum PB-2 were planted in March in Tasmania Australia. The plants were allowed to grow and produce seed capsules. Seed capsules were harvested the following February (ie. at the end of the first growing season).

The seed capsules were dried to a moisture level of 9-16% as determined by a Loss On Drying (LOD) analysis.

Sample of the dried seed capsules from each of the Papaver bracteatum cultivars were finely ground using the laboratory mill. A 3.0 g sample was extracted in 100 mL of 10% acetic acid, stirring for 20 minutes. The sample was analysed using gradient elution HPLC using UV detection at 284 nm, referenced to a certified thebaine standard.

The assays of first year poppy heads from the two Papaver bracteatum cultivars, PB-1 and PB-2 varied from 1.86% to 3.88% anhydrous thebaine by weight of the dried seed capsules.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

1-33. (canceled)

34. A poppy plant, or a progeny mutant or derivative plant thereof, wherein the plant produces seed capsules and an extractable quantity of thebaine in the first growing season after germination.

35. The plant of claim 34, wherein the plant is a perennial poppy plant.

36. The plant of claim 34, wherein the plant is of the species Papaver bracteatum.

37. The plant of claim 34, wherein the plant produces seed capsules and an extractable quantity of thebaine in the first growing season after germination in a temperate poppy growing region.

38. The plant of claim 34, wherein the extractable quantity of thebaine comprises anhydrous thebaine at a concentration of at least 1.0% by weight of a seed capsule of the plant when dried to moisture level of 9-16% as determined by a Loss On Drying (LOD) analysis.

39. The plant of claim 34, wherein the plant is produced from seed deposited with the National Collections of Industrial and Marine Bacteria Limited under accession number NCIMB 41534; or a progeny, mutant or derivative of said plant that retains the ability to produce seed capsules and an extractable quantity of thebaine in the first growing season after germination.

40. The plant of claim 34, wherein the plant is produced from seed deposited with the National Collections of Industrial and Marine Bacteria Limited under accession number NCIMB 41535; or a progeny, mutant or derivative of said plant that retains the ability to produce seed capsules and an extractable quantity of thebaine in the first growing season after germination.

41. The plant of claim 34, wherein the plant produces an indehiscent seed capsule.

42. The plant of claim 34, wherein the plant produces a dehiscent seed capsule.

43. Reproductive material derived from the plant of claim 34.

44. The reproductive material of claim 43, wherein the reproductive material comprises a seed.

45. The reproductive material of claim 44 which is a seed as deposited with the National Collections of Industrial and Marine Bacteria Limited under accession number NCIMB 41534, or a mutant or derivative thereof.

46. The reproductive material of claim 44 which is a seed as deposited with the National Collections of Industrial and Marine Bacteria Limited under accession number NCIMB 41535 or a mutant or derivative thereof.

47. Straw, straw concentrate or latex derived from the plant of claim 34.

48. A method of producing thebaine, the method comprising growing a poppy plant according to claim 34 wherein the plant produces seed capsules and thebaine in the first growing season after germination; and harvesting thebaine from the poppy plant or a part thereof.

49. Thebaine produced according to the method of claim 48.

50. An isolated cell derived from the plant of claim 34.

51. An in-vitro culture comprising one or more of the cells of claim 50.

52. The in-vitro culture of claim 51 wherein said culture produces thebaine.
